(19) United States
(12) Patent Application Publication
(10) Pub. No.: US 2024/0296563 A1
(43) Pub. Date: Sep. 5, 2024

(54) IMAGE PROCESSING APPARATUS AND ENDOSCOPE SYSTEM

(71) Applicant: FUJIFILM Corporation, Tokyo (JP)

(72) Inventor: Toshihiro Usuda, Kanagawa (JP)

(73) Assignee: FUJIFILM Corporation, Tokyo (JP)

(21) Appl. No.: 18/661,680

(22) Filed: May 12, 2024

Related U.S. Application Data
(63) Continuation of application No. PCT/JP2022/039847, filed on Oct. 26, 2022.

(30) Foreign Application Priority Data
Nov. 19, 2021 (JP) 2021-188501

(51) Int. Cl. G06T 7/00; G06F 3/14; G16H 30/20

(52) U.S. Cl. CPC G06T 7/0012; G06F 3/14; G16H 30/20; G06T 2207/10068; G06T 2207/30096

(57) ABSTRACT

The image processing apparatus processes images captured by an endoscope in time series, and includes a recognizer and a processor. The recognizer performs recognition processing on the images. The processor is configured to: acquire the images in a time-series order; cause a display unit to display the images in the time-series order; in response to an instruction for executing the recognition processing, cause the recognizer to perform the recognition processing on, as a first image, an image among the images, which is being displayed on the display unit at a time point of reception of the instruction; cause the display unit to display first information based on a processing result obtained by the recognizer; and store, in a storage unit, third information on the processing result obtained by the recognizer in association with second information for identifying the time point of reception of the instruction for executing the recognition processing.

### CROSS-REFERENCE TO RELATED APPLICATIONS

The present application is a Continuation of PCT International Application No. PCT/JP2022/039847 filed on Oct. 26, 2022 claiming priority under 35 U.S.C § 119(a) to Japanese Patent Application No. 2021-188501 filed on Nov. 19, 2021. Each of the above applications is hereby expressly incorporated by reference, in its entirety, into the present application.

### BACKGROUND OF THE INVENTION

### 1. Field of the Invention

The present invention relates to an image processing apparatus and an endoscope system, and in particular, to an image processing apparatus that processes images captured in time series by an endoscope and an endoscope system including the image processing apparatus.

### 2. Description of the Related Art

In recent years, in the medical field, technology development of computer-aided diagnosis (CAD) has been accelerated. In particular, CAD technology development utilizing artificial intelligence (AI) is being accelerated. In general, AI is generated by machine learning such as deep learning.

WO2020/110214A describes a technique of automatically detecting a lesion part in an image, captured by an endoscope, by using a recognizer constituted by a trained model. In addition, WO2018/105063A describes a technique of performing discrimination and classification of a lesion part in an image, captured by an endoscope, by using a recognizer constituted by a trained model.

### SUMMARY OF THE INVENTION

Typically, a processing result obtained by a recognizer is displayed on a display device that displays an image captured by an endoscope. However, the processing result of an image that is being displayed, obtained by the recognizer, is displayed with a delay. Thus, for example, if the processing result of the image that is being displayed, obtained by the recognizer, is stored by screen capturing or the like, there is a problem in that a correct processing result may not be stored.

An embodiment according to the technology of the present disclosure provides an image processing apparatus and an endoscope system that can appropriately store a processing result obtained by a recognizer.
(1) An image processing apparatus that processes images captured in time series by an endoscope, the image processing apparatus including: a recognizer configured to perform recognition processing on the images that are input; and a processor configured to: acquire the images in a time-series order; cause a display unit to display the images in the time-series order; in response to an instruction for executing the recognition processing, cause the recognizer to perform the recognition processing on, as a first image, an image among the images, which is being displayed on the display unit at a time point of reception of the instruction; cause the display unit to display first information based on a processing result obtained by the recognizer; and store, in a storage unit, third information on the processing result obtained by the recognizer in association with second information for identifying the time point of reception of the instruction for executing the recognition processing.
(2) The image processing apparatus according to (1), in which the instruction for executing the recognition processing also serves as an instruction for capturing a still image, and the processor is configured to store the first image in the storage unit in response to the instruction for executing the recognition processing.
(3) The image processing apparatus according to (2), in which the second information is constituted by the first image stored in the storage unit in response to the instruction for executing the recognition processing.
(4) The image processing apparatus according to (1) or (2), in which the second information is constituted by information of a time or a date and time at the time point of reception of the instruction for executing the recognition processing or information of an elapsed time from start of image capturing.
(5) The image processing apparatus according to any one of (1) to (3), in which the processor is configured to: in response to the instruction for executing the recognition processing, store, in the storage unit in association with the second information, the first information being displayed on the display unit at the time point of reception of the instruction; and after the processing result of the first image obtained by the recognizer is output, update the first information stored in the storage unit, based on the processing result obtained by the recognizer, and store the updated first information in the storage unit as the third information.
(6) The image processing apparatus according to (5), in which the third information includes information indicating that the first information is updated.
(7) The image processing apparatus according to (1), in which the processor is configured to: store, in the storage unit, an image that is an image of a screen being displayed on the display unit at the time point of reception of the instruction for executing the recognition processing and that includes the first image and the first information, as a second image; and after the processing result of the first image obtained by the recognizer is output, by updating part of the first information in the second image stored in the storage unit, based on the processing result obtained by the recognizer, set the updated part of the first information as the third information and store, in the storage unit, the second image including part of the first image as the second information.
(8) The image processing apparatus according to (7), in which the processor is configured to cause the first image to be displayed in a first region set within a screen of the display unit, and the first information to be displayed in a second region set in a region different from the first region.
(9) The image processing apparatus according to (7) or (8), in which the third information includes information indicating that the first information is updated.
(10) The image processing apparatus according to any one of (1) to (9), in which the first information and the third information are constituted by an image generated based on the processing result obtained by the recognizer.
(11) The image processing apparatus according to any one of (1) to (9), in which the first information is constituted by an image generated based on the processing result obtained by the recognizer, and the third information is constituted by information necessary for generating an image constituting the first information.
(12) The image processing apparatus according to any one of (1) to (9), in which the third information is constituted by information necessary for generating the first information.
(13) The image processing apparatus according to (11) or (12), in which the third information is information indicating the processing result obtained by the recognizer.
(14) The image processing apparatus according to any one of (1) to (13), in which the recognizer is configured to detect a lesion part, and the first information is constituted by information indicating a position of the lesion part.
(15) The image processing apparatus according to any one of (1) to (13), in which the recognizer is configured to discriminate a lesion part, and the first information is constituted by information indicating a discrimination result of the lesion part.
(16) The image processing apparatus according to any one of (1) to (13), in which the recognizer is configured to determine a type of a lesion part, and the first information is constituted by information indicating the type of the lesion part.
(17) The image processing apparatus according to any one of (1) to (13), in which the recognizer is configured to determine a site of a specific organ, and the first information is constituted by information indicating an observed site in the organ.
(18) An image processing apparatus that processes images captured in time series by an endoscope, the image processing apparatus including: a recognizer configured to perform recognition processing on the images that are input; and a processor configured to: acquire the images in a time-series order; cause a display unit to display the images in the time-series order; in response to an instruction for executing the recognition processing, cause the recognizer to perform the recognition processing on, as a first image, an image among the images, which is being displayed on the display unit at a time point of reception of the instruction; cause the display unit to display first information based on a processing result obtained by the recognizer; acquire, as a second image, an image that is an image of a screen displayed on the display unit at the time point of reception of the instruction for executing the recognition processing and that includes the first image and the first information; generate, as a third image, an image in which part of the first information in the second image is replaced with fourth information; and store the third image in a storage unit.
(19) An endoscope system including: an endoscope; and the image processing apparatus according to any one of (1) to (18).

According to the present invention, a processing result obtained by a recognizer can be appropriately stored.

### BRIEF DESCRIPTION OF THE DRAWINGS

FIG. 1 is a block diagram illustrating an example of a system configuration of an endoscope system;

FIG. 2 is a block diagram of main functions of a processor device;

FIG. 3 is a block diagram illustrating an example of a hardware configuration of an image processing apparatus;

FIG. 4 is a block diagram of main functions of the image processing apparatus;

FIG. 5 is a block diagram of main functions of a recognition processing unit;

FIG. 6 is a diagram illustrating an example of observation state determination results;

FIG. 7 is a diagram illustrating an example of screen display;

FIG. 8 is a diagram illustrating an example of an observation state display map;

FIG. 9 is a diagram illustrating an overview of a process flow in a case where information of an observation state determination result is recorded;

FIG. 10 is a diagram illustrating an example of imaging determination criteria set for each observation target site;

FIG. 11 is a block diagram of main functions of the image processing apparatus;

FIG. 12 is a conceptual diagram of storage processing;

FIG. 13 is a block diagram of main functions of the image processing apparatus;

FIG. 14 is a conceptual diagram of storage processing;

FIG. 15 is a diagram illustrating an example of a storage screen image in a case where an observation state display map is updated;

FIGS. 16A and 16B are diagrams illustrating an example of replacement with another image; and

FIGS. 17A and 17B are diagrams illustrating an example of a process in a case where a discrimination result is stored by screen capturing.

### DESCRIPTION OF THE PREFERRED EMBODIMENTS

Hereinafter, preferred embodiments of the present invention will be described in detail with reference to the accompanying drawings.

### First Embodiment

Here, a case will be described as an example in which the present invention is applied to an endoscope system that performs an endoscopic examination of an upper digestive organ, in particular, a stomach.

### Configuration of Endoscope System

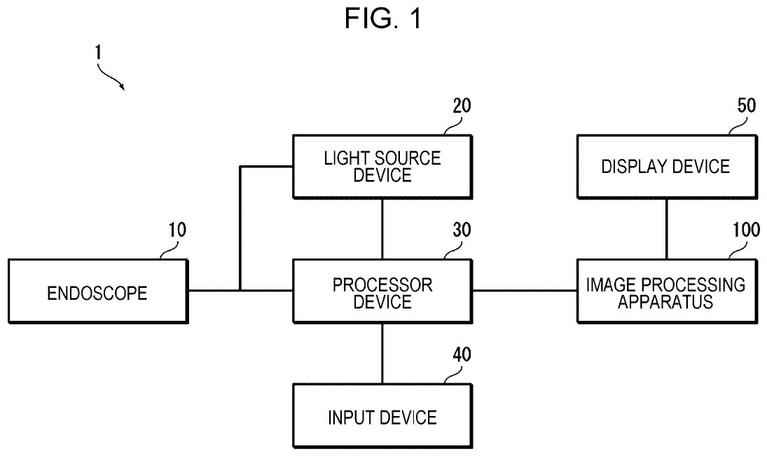

FIG. 1 is a block diagram illustrating an example of a system configuration of the endoscope system.

As illustrated in FIG. 1, an endoscope system 1 according to this embodiment includes an endoscope 10, a light source device 20, a processor device 30, an input device 40, a display device 50, an image processing apparatus 100, and the like. The endoscope 10 is connected to the light source device 20 and the processor device 30. The light source device 20, the input device 40, and the image processing apparatus 100 are connected to the processor device 30. The display device 50 is connected to the image processing apparatus 100.

The endoscope system 1 according to this embodiment is configured as a system by which observation using special light (special-light observation) is possible in addition to observation using normal white light (white-light observation). The special-light observation includes narrow-band light observation. The narrow-band light observation includes blue laser imaging observation (BLI observation), narrow band imaging observation (NBI observation), linked color imaging observation (LCI observation), and the like. Note that the special-light observation itself is a known technique, and thus, detailed description thereof is omitted.

### Endoscope

The endoscope 10 according to this embodiment is an electronic endoscope (flexible endoscope), in particular, an electronic endoscope for the upper digestive organ. The electronic endoscope includes an operating unit, an insertion unit, a connection unit, and the like, and images a subject with an imaging element incorporated in a distal end of the insertion unit. As the imaging element, a color image pickup element (e.g., a color image pickup element using a complementary metal oxide semiconductor (CMOS), a charge coupled device (CCD), or the like) having predetermined filter arrangement (e.g., Bayer arrangement) is used. The operating unit includes an angle knob, an air/water supply button, a suction button, a mode switching button, a release button, a forceps port, and the like. The mode switching button is a button for switching an observation mode. For example, switching is performed among a mode for white-light observation, a mode for LCI observation, and a mode for BLI observation. The release button is a button for issuing an instruction for capturing a still image. Note that the endoscope itself is known, and thus, detailed description thereof is omitted. The endoscope 10 is connected to the light source device 20 and the processor device 30 via the connection unit.

### Light Source Device

The light source device 20 generates illumination light to be supplied to the endoscope 10. As described above, the endoscope system 1 according to this embodiment is configured as a system by which the special-light observation is possible in addition to the normal white-light observation. Thus, the light source device 20 has a function of generating light (e.g., narrow-band light) compatible with the special-light observation in addition to the normal white light. Note that, as described above, the special-light observation itself is a known technique, and thus, description of generation of the illumination light is omitted. The light source type is switched, for example, by the mode switching button provided in the operating unit of the endoscope 10.

### Processor Device

The processor device 30 integrally controls the operation of the entire endoscope system. The processor device 30 includes, as its hardware configuration, a processor, a main memory, an auxiliary storage, an input/output interface, and the like.

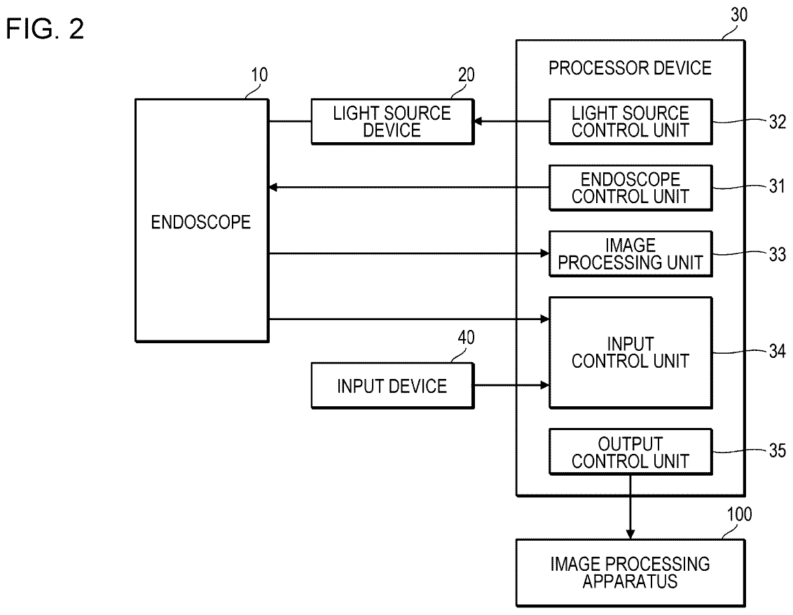

FIG. 2 is a block diagram of main functions of the processor device 30.

As illustrated in FIG. 2, the processor device 30 has functions of an endoscope control unit 31, a light source control unit 32, an image processing unit 33, an input control unit 34, an output control unit 35, and the like. Each function is implemented by the processor executing a predetermined program. The auxiliary storage stores various programs to be executed by the processor, various kinds of data necessary for control, and the like.

The endoscope control unit 31 controls the endoscope 10. The control of the endoscope 10 includes driving control of the imaging element, control of air/water supply, control of suction, and the like.

The light source control unit 32 controls the light source device 20. The control of the light source device 20 includes light emission control of the light source, switching control of the light source type, and the like.

The image processing unit 33 performs processing of generating a captured image by performing various kinds of signal processing on a signal output from the imaging element of the endoscope 10.

The input control unit 34 performs processing of receiving an input of an operation from the input device 40 and the operating unit of the endoscope 10 and an input of various kinds of information.

The output control unit 35 controls output of information to the image processing apparatus 100. The information output to the image processing apparatus 100 includes, in addition to an image captured by the endoscope (endoscopic image), information input through the input device 40, various kinds of operation information, and the like. The various kinds of operation information include operation information of the operating unit of the endoscope 10 in addition to operation information of the input device 40. The operation information includes an instruction for capturing a still image. The instruction for capturing a still image is issued, for example, by a button operation of a release button provided in the operating unit in the endoscope 10. In addition, the instruction for capturing an image may be issued through a foot switch, an audio input device, a touch panel, or the like.

### Input Device

The input device 40 constitutes a user interface in the endoscope system 1 together with the display device 50. The input device 40 includes, for example, a keyboard, a mouse, a foot switch, or the like. In addition, the input device 40 may also include a touch panel, a voice input device, a line-of-sight input device, or the like.

### Display Device

The display device 50 is used to display an endoscopic image and also to display various kinds of information. The display device 50 includes, for example, a liquid crystal display (LCD), an organic electroluminescence display (OLED), or the like. In addition, the display device 50 may also include a projector, a head-mounted display, or the like. In this embodiment, the display device 50 is an example of a display unit.

### Image Processing Apparatus

The image processing apparatus 100 performs various kinds of recognition processing on an image captured by the endoscope 10. As an example, in this embodiment, processing of detecting a lesion part in an image, processing of discriminating the detected lesion part, processing of determining an observation state, and the like are performed. In addition, the image processing apparatus 100 performs processing of outputting the image captured by the endoscope 10, including a result of the recognition processing, to the display device 50. Furthermore, the image processing apparatus 100 performs processing of capturing and recording a still image in accordance with an instruction from a user.

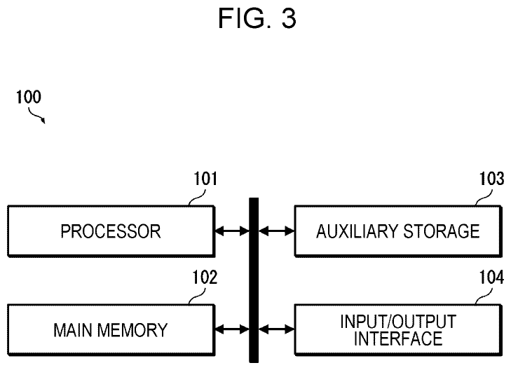

FIG. 3 is a block diagram illustrating an example of a hardware configuration of the image processing apparatus.

The image processing apparatus 100 is constituted by a so-called computer and includes, as its hardware configuration, a processor 101, a main memory 102, an auxiliary storage 103, an input/output interface 104, and the like. The image processing apparatus 100 is connected to the processor device 30 and the display device 50 via the input/output interface 104. The auxiliary storage 103 is constituted by, for example, a flash memory or the like including a hard disk drive (HDD) or a solid state drive (SSD). The auxiliary storage 103 stores programs to be executed by the processor 101 and various kinds of data necessary for control or the like. Images (still image and moving image) captured by the endoscope and results of the recognition processing are stored in the auxiliary storage 103.

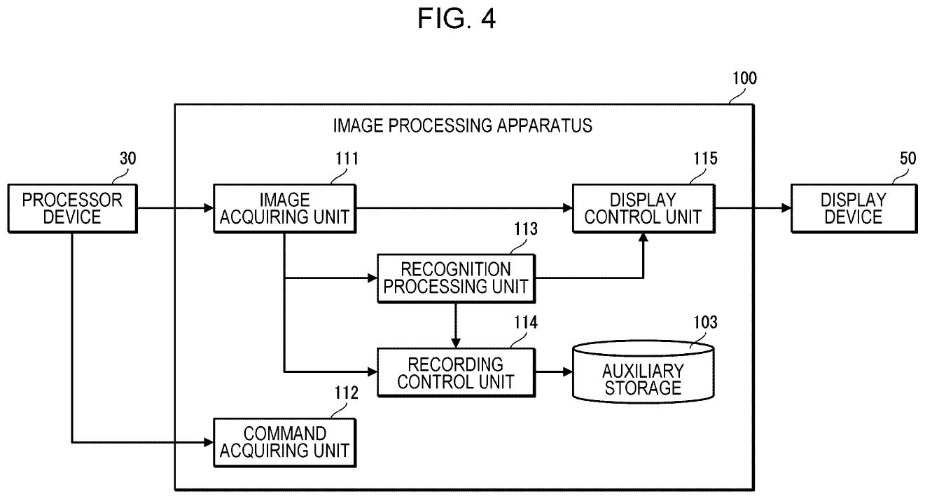

FIG. 4 is a block diagram of main functions of the image processing apparatus.

As illustrated in FIG. 4, the image processing apparatus 100 has functions of an image acquiring unit 111, a command acquiring unit 112, a recognition processing unit 113, a recording control unit 114, a display control unit 115, and the like. The function of each unit is implemented by the processor 101 executing a predetermined program (image processing program).

The image acquiring unit 111 performs processing of acquiring, in a time-series order, images captured in time series by the endoscope 10. The images are acquired via the processor device 30.

The command acquiring unit 112 acquires command information. The command information includes information of an instruction for capturing a still image. As described above, the instruction for capturing a still image is issued by the release button provided in the operating unit of the endoscope 10.

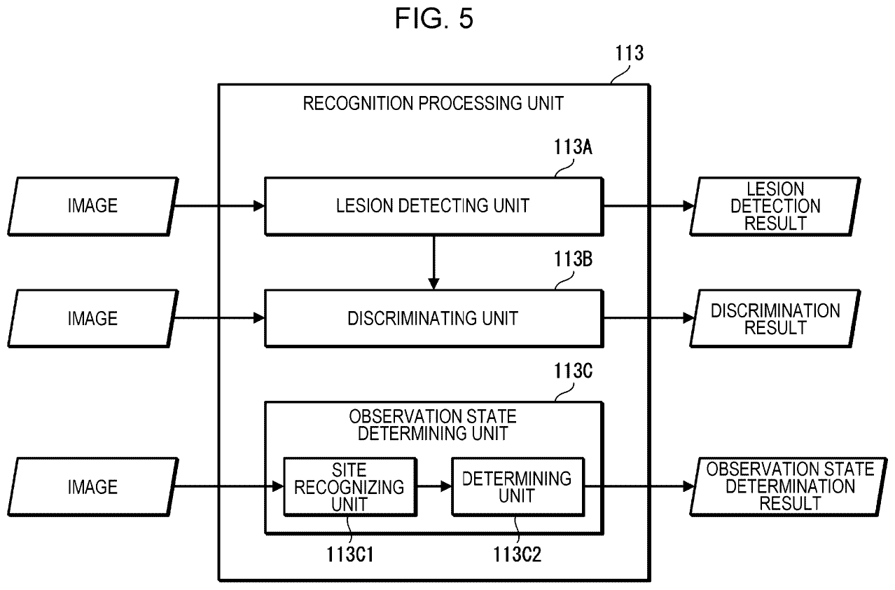

The recognition processing unit 113 performs various kinds of processing by performing image recognition on the images acquired by the image acquiring unit 111. FIG. 5 is a block diagram of main functions of the recognition processing unit. As illustrated in FIG. 5, the recognition processing unit 113 according to this embodiment has functions of a lesion detecting unit 113A, a discriminating unit 113B, an observation state determining unit 113C, and the like.

The lesion detecting unit 113A performs image recognition on an input image to detect a lesion part such as a polyp included in the image. The lesion part includes, in addition to a part that is definitely a lesion part, a part that may be a lesion (e.g., benign tumor or dysplasia), a part having a feature that may be directly or indirectly related to a lesion (e.g., redness), and the like. The lesion detecting unit 113A is constituted by a trained model that is trained to recognize a lesion part in an image. The detection of the lesion part using the trained model itself is a known technique, and thus, detailed description thereof is omitted. As an example, the lesion detecting unit 113A is constituted by a model using a convolutional neural network (CNN). Note that the detection of the lesion part can include determination of the type of the detected lesion part.

The discriminating unit 113B performs discrimination processing on the lesion part detected by the lesion detecting unit 113A. As an example, in this embodiment, the discriminating unit 113B performs processing of discriminating between neoplastic (NEOPLASTIC) and non-neoplastic (HYPERPLASTIC) for the lesion part such as a polyp detected by the lesion detecting unit 113A. The discriminating unit 113B is constituted by a trained model that is trained to discriminate a lesion part in an image.

The observation state determining unit 113C performs processing of determining an observation state of an examination target. Specifically, the observation state determining unit 113C performs processing of determining whether a predetermined site of the examination target is observed. In this embodiment, whether the predetermined site is observed is determined based on a captured still image. That is, it is determined whether the predetermined site is included in the captured still image to determine whether the predetermined site of the examination target is observed. Thus, a still image is assumed to be captured. The predetermined site (observation target site) is determined for each examination target in accordance with an examination purpose or the like. For example, when the examination target is a stomach, as examples, set observation target sites are (1) esophagogastric junction, (2) lesser curvature immediately below cardia (imaged by J-turn operation), (3) greater curvature immediately below cardia (imaged by U-turn operation), (4) lesser curvature posterior wall from angulus or lower body part (imaged by J-turn operation), (5) pyloric ring from prepyloric region, and (6) greater curvature in lower body part from above. These sites have to be intentionally recorded. In addition, intentional endoscope operations are required at these sites. Note that (4) “lesser curvature posterior wall from angulus or lower body part” may also be “lesser curvature in lower body part (imaged by J-turn operation)” in consideration of the fact that it is sometimes not possible to image the gastric angle and the fact that it is not possible to reliably image the posterior wall. In addition, (5) “pyloric ring from prepyloric region” may also be “entire view of antrum” in which importance is attached to whether the antrum is imaged in a bird's eye view rather than imaging the pyloric ring in a pinpoint manner. In addition, (6) “greater curvature in lower body part from above” is not limited to the lower body part, and may be “greater curvature from above” in which importance is attached to the fact that the greater curvature with the folds open is imaged.

The observation state determining unit 113C includes a site recognizing unit 113C1 and a determining unit 113C2. The site recognizing unit 113C1 recognizes a site in an image. Based on a recognition result obtained by the site recognizing unit 113C1, the determining unit 113C2 determines whether the observation target site is observed (imaged).

The site recognizing unit 113C1 performs image recognition on an input image to perform processing of recognizing a site included in the image. The site recognizing unit 113C1 is constituted by a trained model that is trained to recognize a site in an image. As an example, the site recognizing unit 113C1 is constituted by a CNN. In this embodiment, the trained model constituting the site recognizing unit 113C1 is an example of a recognizer.

As described above, in this embodiment, whether the observation target site is observed is determined based on the captured still image. Thus, the still image is input to the site recognizing unit 113C1. The still image is captured in accordance with an instruction for capturing an image from a user. Thus, in this embodiment, the instruction for capturing a still image also serves as an instruction for executing observation state determination processing.

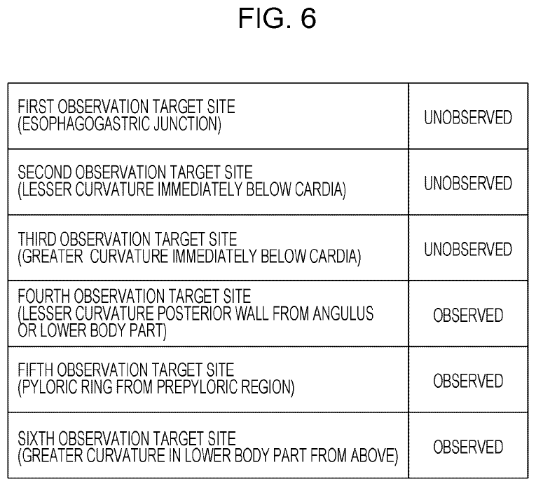

Based on a recognition result obtained by the site recognizing unit 113C1, the determining unit 113C2 determines whether the observation target site is observed (imaged). In this embodiment, it is determined whether the above six sites are observed. FIG. 6 is a diagram illustrating an example of observation state determination results. As illustrated in FIG. 6, as the determination results, whether the respective observation target sites are “observed” or “unobserved” is indicated. An observation target site recognized at least once is regarded as “observed”. On the other hand, an observation target site that is yet to be recognized is regarded as “unobserved”. Information of the observation state determination results constitutes information necessary for generating an observation state display map. The observation state display map illustrates the observation state by a diagram. Details of the observation state display map will be described later.

In response to the instruction for capturing a still image, the recording control unit 114 performs processing of capturing a still image and recording (storing) it in the auxiliary storage 103. Furthermore, if an observation state determination function is ON, the recording control unit 114 performs processing of recording (storing), in the auxiliary storage 103, the information of the observation state determination results in association with the still image.

As the still image, an image being displayed on the display device 50 at a time point of reception of the instruction for capturing a still image is stored. Thus, the user can store a desired image as a still image. In response to the instruction for capturing a still image, the recording control unit 114 acquires an image of a frame being displayed on the display device 50 and records the image in the auxiliary storage 103. If the observation state determination function is ON, the image of the frame (image to be recorded as still image) is input to the observation state determining unit 113C. The recording control unit 114 acquires the processing result and records it in the auxiliary storage 103 in association with the still image. In this embodiment, the still image recorded in the auxiliary storage 103 is an example of second information for identifying a time point at which an instruction for executing the recognition processing is received. In addition, the information of the observation state determination results recorded in the auxiliary storage 103 in association with the still image is an example of third information. Furthermore, the auxiliary storage 103 is an example of a storage unit.

Note that the association method is not limited to a particular method. It is only necessary to record the still image and the information of the observation state determination results based on the still image in a form in which a correspondence relationship therebetween can be understood. Thus, for example, the association between the two may be managed by a separately generated management file. In addition, for example, the information of the observation state determination results may be recorded as accessory information (so-called meta information) of the still image.

The display control unit 115 controls display on the display device 50. The display control unit 115 causes the display device 50 to display, in a time-series order, images captured in time series by the endoscope 10. In addition, the display control unit 115 causes the display device 50 to display information based on the result of the recognition processing performed by the recognition processing unit 113.

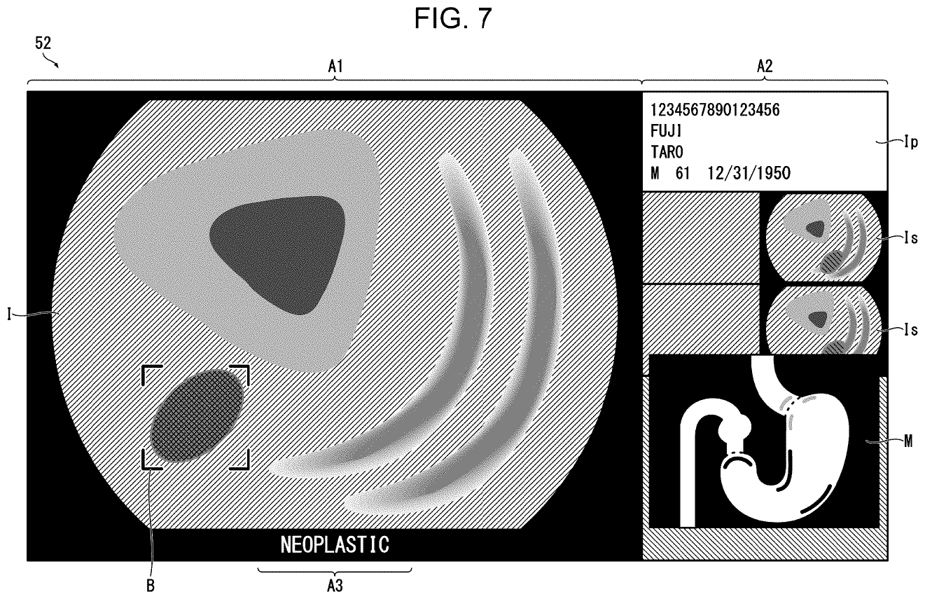

FIG. 7 is a diagram illustrating an example of screen display. FIG. 7 illustrates an example of a case where the display device 50 is a so-called wide monitor (monitor with horizontally long screen).

As illustrated in FIG. 7, an image I captured by the endoscope is displayed in real time in a main display region A1 set within a screen 52. That is, a live view is displayed. The main display region A1 is an example of a first region. A sub-display region A2 is further set on the screen 52, and various kinds of information related to the examination are displayed. In the example illustrated in FIG. 7, an example in a case where information Ip on a patient and still images Is captured during the examination are displayed in the sub-display region A2 is illustrated. The still images Is are displayed, for example, in the order the images are captured from top to bottom of the screen 52.

In addition, as illustrated in FIG. 7, if a lesion part detection support function is ON, a lesion part detection result is displayed on the screen 52. The lesion part detection result is displayed in a form in which the detected lesion part is surrounded by a frame (so-called bounding box) B. The frame B is an example of information indicating the position of the lesion part. In a case where the type of the lesion part is determined by the lesion detecting unit 113A, information of the determined type of the lesion part is displayed on the screen in place of or in addition to the information indicating the position of the lesion part. The information of the type of the lesion part is displayed at a predetermined position on the screen, for example, near the detected lesion part or in the sub-display region A2.

In addition, as illustrated in FIG. 7, if a discrimination support function is ON, a discrimination result is displayed on the screen 52. The discrimination result is displayed in a discrimination result display region A3 set within the screen 52. FIG. 7 illustrates an example of a case where the discrimination result is “neoplastic (NEOPLASTIC)”.

Furthermore, as illustrated in FIG. 7, if an observation state determination function is ON, an observation state display map M indicating the observation state is displayed on the screen 52. The observation state display map M is generated based on the observation state determination results and is displayed at a predetermined position. This position is set to a position at which the display does not overlap with the image I captured by the endoscope. FIG. 7 illustrates an example of a case where the observation state display map M is displayed in the sub-display region A2. In the sub-display region A2, the observation state display map M is displayed with priority over other displays. That is, if the observation state display map M overlaps with another piece of information, the observation state display map M is displayed at the top. In this embodiment, the region in which the observation state display map M is displayed is an example of a second region. This region is different from the region in which the image I captured by the endoscope is displayed.

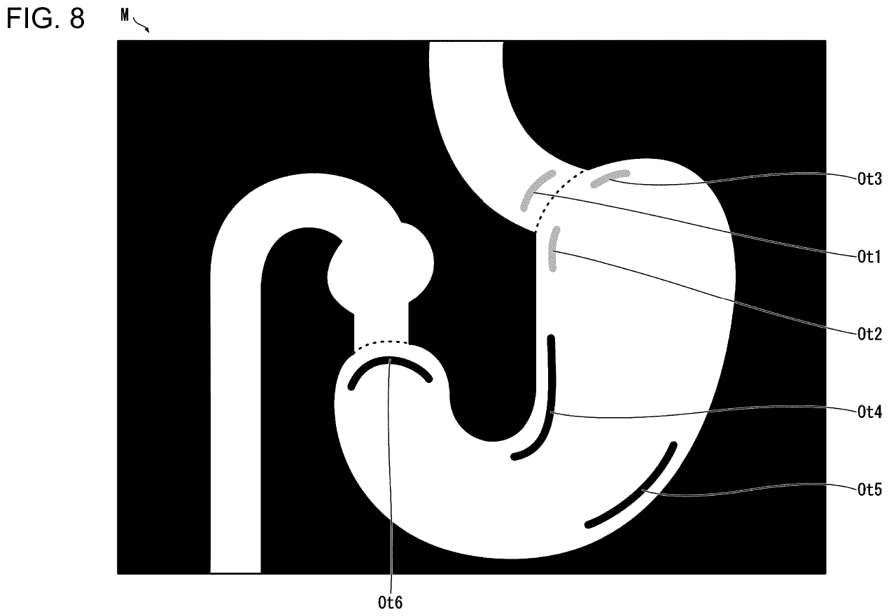

FIG. 8 is a diagram illustrating an example of the observation state display map. FIG. 8 illustrates an example of a case where the examination target is a stomach.

As illustrated in FIG. 8, the observation state display map M is generated using a schema diagram of an organ that is the examination target. In this embodiment, a schema diagram of a stomach is used for generation. Specifically, as illustrated in FIG. 8, a schema diagram of the stomach is displayed in a rectangular box, and observation target sites Ot1 to Ot6 are indicated by lines on the schema diagram. FIG. 8 illustrates an example of a case where the first observation target site Ot1 is “esophagogastric junction”, the second observation target site Ot2 is “lesser curvature immediately below cardia”, the third observation target site Ot3 is “greater curvature immediately below cardia”, the fourth observation target site Ot4 is “lesser curvature posterior wall from angulus or lower body part”, the fifth observation target site Ot5 is “pyloric ring from prepyloric region”, and the sixth observation target site Ot6 is “greater curvature in lower body part from above”.

The lines indicating the observation target sites Ot1 to Ot6 are displayed in different colors depending on whether the observation target sites are “observed” or “unobserved”. For example, the line of the “unobserved” observation target site is displayed in gray, and the line of the “observed” observation target site is displayed in green (displayed in black in FIG. 8). FIG. 8 illustrates an example of a case where the first observation target site Ot1, the second observation target site Ot2, and the third observation target site Ot3 are “unobserved”, and the fourth observation target site Ot4, the fifth observation target site Ot5, and the sixth observation target site Ot6 are “observed”.

In this manner, by displaying the lines indicating the observation target sites in different colors, it is possible to grasp at a glance whether the observation target sites are observed or yet to be observed. In this embodiment, the observation state display map M is an example of first information.

### Operation of Endoscope System

As a basic operation of the endoscope system 1, the image processing apparatus 100 causes the display device 50 to display an image captured by the endoscope 10 in real time.

In addition to this basic operation, the image processing apparatus 100 causes the display device 50 to display various kinds of support information. In this embodiment, as the support information, information of a detection result of a lesion part, information of a discrimination result of the lesion part, and information of an observation state determination result are displayed. Each piece of support information is displayed if the corresponding function is ON. For example, if the lesion part detection support function is ON, a detected lesion part is displayed while being surrounded by a frame as the detection result of the lesion part. In addition, if the discrimination support function is ON, the discrimination result is displayed in the discrimination result display region A3. Furthermore, if the observation state determination function is ON, the observation state display map M is displayed on the screen 52 as the observation state determination result. The observation state determination processing is performed based on a captured still image. That is, it is determined whether a predetermined observation target site is observed by determining whether the observation target site is included in the captured still image.

Furthermore, in response to an instruction for capturing a still image from a user, the image processing apparatus 100 records, as a still image, an image of a frame being displayed on the display device 50.

If the observation state determination function is ON, the image processing apparatus 100 records information of the observation state determination result in association with the captured still image.

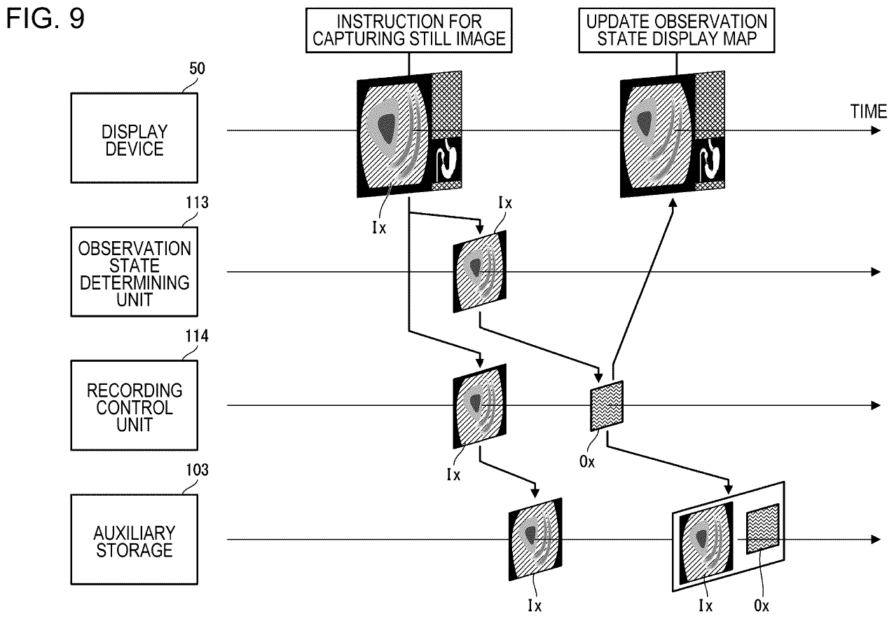

FIG. 9 is a diagram illustrating an overview of a process flow in a case where the information of the observation state determination result is recorded.

As illustrated in FIG. 9, the image I captured by the endoscope is displayed on the display device 50 at a predetermined frame rate.

When an instruction for capturing a still image is issued at a specific time point, an image Ix being displayed on the display device 50 at the time point of reception of the instruction is acquired by the recording control unit 114 and recorded in the auxiliary storage 103. The image Ix is input to the observation state determining unit 113C. The observation state determining unit 113C processes the input image and outputs an observation state determination result. The recording control unit 114 acquires information Ox of the observation state determination result output from the observation state determining unit 113C, and records the information Ox in the auxiliary storage 103 in association with the previously recorded still image Ix. The information Ox of the observation state determination result is constituted by information indicating a determination result of “observed” or “unobserved” for each observation target site.

The information Ox of the observation state determination result is also output to the display control unit 115. Based on the acquired information Ox of the observation state determination result, the display control unit 115 generates the observation state display map M and displays the observation state display map M at a predetermined position on the screen 52 (see FIG. 7).

In this manner, according to the endoscope system 1 according to this embodiment, it is possible to store a still image and an observation state determination result based on the still image in appropriate association with each other. Thus, for example, when the still image is checked after an examination, an accurate observation state can be referred to.

The observation state display map M is displayed on the screen 52 of the display device 50 (see FIG. 7), but the display is updated with delay. Thus, for example, if the image I captured by the endoscope and the observation state display map M at that time are stored by screen capturing or the like, the image I and the observation state display map M that do not have an accurate correspondence relationship are stored. In contrast, since the endoscope system 1 according to this embodiment acquires and stores an observation state determination result based on a captured still image, the still image and the observation state determination result can be stored in an accurate correspondence relationship.

### Modifications

### Storage of Information of Observation State Determination Result

The above embodiment adopts a configuration in which a captured still image and information of an observation state determination result based on the still image are stored in association with each other. However, it is also possible to adopt a configuration in which only information of an observation state determination result at a specific time point is stored. For example, it is possible to adopt a configuration in which observation state determination processing is performed at a time point of reception of an instruction for storing the observation state determination result (instruction for executing recognition processing), and the result is stored. In this case, an image being displayed on the display device at the time point of reception of the instruction for storing the observation state determination result is input to the observation state determining unit 113C, and the result is acquired and recorded in the auxiliary storage 103. In this case, result information is recorded in association with time information or date and time information at a time point of reception of a storage instruction. Alternatively, the result information is recorded in association with information of an elapsed time from the start of imaging at the time point of reception of the storage instruction.

In addition, also in a case of recording in association with a still image, instead of recording in direct association with the still image, it is possible to adopt a configuration in which recording is performed in indirect association with the still image. For example, in a case where the result information is recorded in association with the date and time information or time information as described above, the still image is also recorded in association with the date and time information or time information. Thus, both can be associated with each other based on the date and time or the time. The same applies to a case of recording in association with information of an elapsed time from the start of imaging.

In addition, the information of the observation state determination result recorded in association with the still image is more preferably recorded by including information indicating that the information is updated. Thus, it is possible to confirm with ease that the information is updated.

### Information to be Stored

The above embodiment adopts a configuration in which, in association with a still image, information of an observation state determination result based on the still image is stored. However, the information to be stored in association with the still image is not limited to this. Information of a detection result and/or information of a discrimination result of a lesion part may also be stored. For example, in a case where the information of the detection result of the lesion part is stored in association with a captured still image, in response to an instruction for capturing a still image, the image being displayed on the display device is stored and input to the lesion detecting unit. Then, the information of the detection result of the lesion part obtained by the lesion detecting unit is acquired and stored in association with the still image. In addition, for example, in a case where the information of the discrimination result of the lesion part is stored in association with the captured still image, in response to the instruction for capturing a still image, the image being displayed on the display device is stored and input to the discriminating unit. Then, the information of the discrimination result obtained by the discriminating unit is acquired and stored in association with the still image.

### Determination of Observation State

The above embodiment adopts a method of determining an observation state depending on whether an image of a preset observation target site is captured as a still image. However, the method of determining an observation state is not limited to this. It is also possible to adopt a method of determining an observation state by determining whether an observation target site is included in an image captured by an endoscope.

Regarding whether an image of an observation target site is captured, whether the image quality is good or bad may also be added to determination conditions. Whether the image quality is good or bad is determined for each of divided elements such as, for example, out-of-focus, blurring, brightness, composition, dirt, presence or absence of a target region, and the like. In addition, whether the image quality is good or bad is set for each of observation target sites, and if a captured image satisfies all requirements, it is determined that the observation target site is imaged.

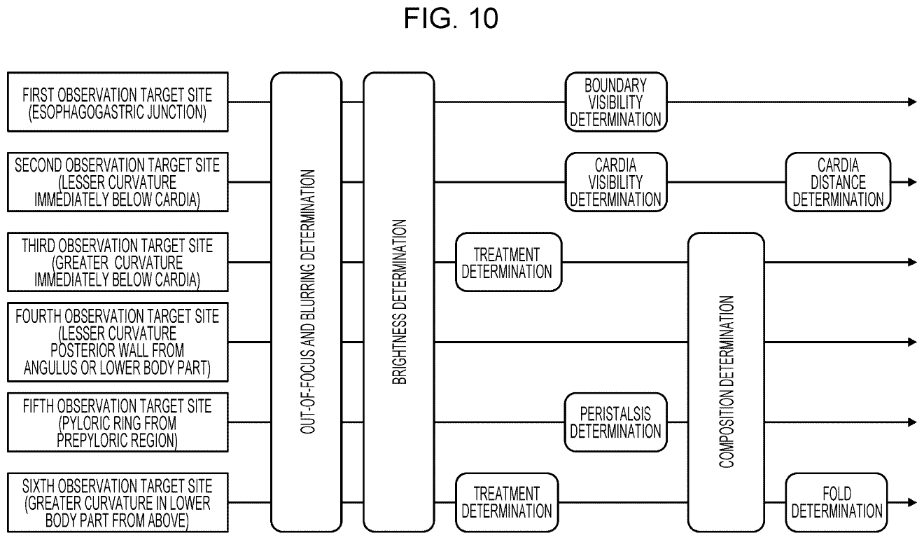

FIG. 10 is a diagram illustrating an example of imaging determination criteria set for each observation target site.

As illustrated in FIG. 10, out-of-focus and blurring determination of an image is performed for all observation target sites. In addition, brightness determination of an image is performed for all the observation target sites.

In addition, if the observation target site is the esophagogastric junction (first observation target site), boundary visibility determination is further performed. The boundary visibility determination determines whether the junction between the stomach and the esophagus is visible in an image.

In addition, if the observation target site is the lesser curvature immediately below the cardia (second observation target site), cardia visibility determination and cardia distance determination are further performed. The cardia visibility determination determines whether the cardia is visible in the image. The cardia distance determination measures the distance to the cardia (imaging distance) in the image, and determines whether the distance is appropriate.

In addition, if the observation target site is the greater curvature immediately below the cardia (third observation target site), treatment determination and composition determination are further performed. The treatment determination determines whether water, a residue, foam, or the like is accumulated in the observation target site, based on the image. The composition determination determines whether an imaging composition is appropriate. For example, it is determined whether the observation target site is captured at the center of the image.

In addition, if the observation target site is the lesser curvature posterior wall from the angulus or lower body part (fourth observation target site), composition determination is further performed.

In addition, if the observation target site is the pyloric ring from the prepyloric region (fifth observation target site), peristalsis determination and composition determination are further performed. The peristalsis determination determines whether peristalsis is present in the observation target site, based on the image.

In addition, if the observation target site is the greater curvature in the lower body part from above (sixth observation target site), treatment determination, composition determination, and fold determination are further performed. The fold determination determines whether a fold of the observation target site extends in the image.

A determiner that performs each determination can be constituted by, for example, a trained model.

### Second Embodiment

The first embodiment described above adopts a configuration in which, in association with a captured still image, information of an observation state determination result based on the still image is stored. In this embodiment, in association with a captured still image, an observation state display map based on the still image is stored.

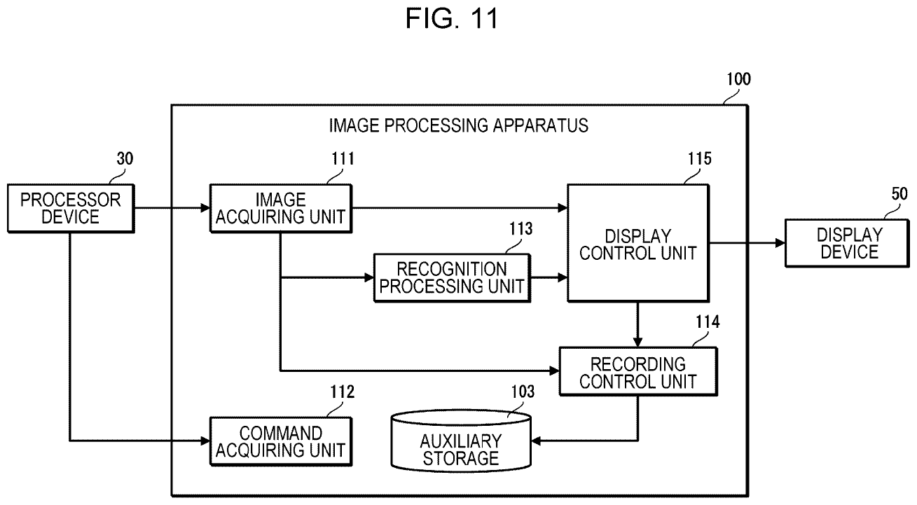

FIG. 11 is a block diagram of main functions of the image processing apparatus.

In response to an instruction for capturing a still image, the recording control unit 114 acquires an image of a frame being displayed on the display device 50 at a time point of the instruction for capturing an image, and stores the image in the auxiliary storage 103. The stored image constitutes a captured still image. In this embodiment, the stored still image is an example of a first image and is an example of second information.

The recognition processing unit 113 includes the observation state determining unit 113C (see FIG. 5). In response to the instruction for capturing a still image, an image of a frame being displayed on the display device 50 at the time point of the instruction for capturing an image is input to the observation state determining unit 113C. Based on an observation state determination result obtained by the observation state determining unit 113C, an observation state display map is generated and displayed on the display device 50.

The recording control unit 114 acquires the generated observation state display map and stores it in the auxiliary storage 103 in association with the captured still image. The observation state display map is acquired in the form of image data. In this embodiment, the observation state display map is an example of first information and third information.

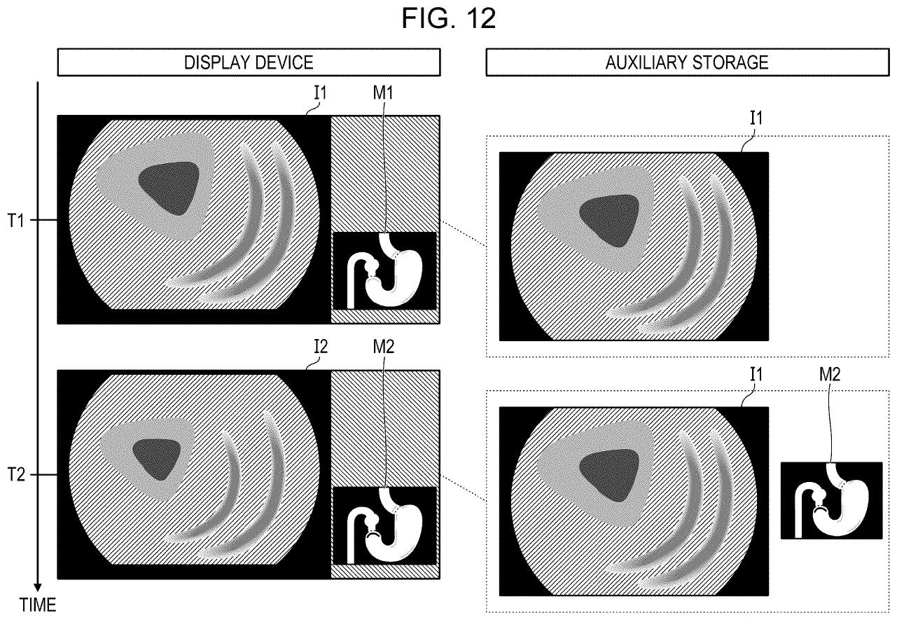

FIG. 12 is a conceptual diagram of storage processing.

It is assumed that an instruction for capturing a still image is issued at time T1. In response to the instruction for capturing an image, an image I1 being displayed on the display device 50 at that time point (time T1) is stored in the auxiliary storage 103.

Here, an observation state display map M1 displayed on the display device 50 at time T1 is in a state in which a determination result is yet to be reflected. For example, it is assumed that image capturing at time T1 is initial image capturing. In this case, the observation state display map M1 displayed on the display device 50 at time T1 indicates that all observation target sites are in an unobserved state. That is, the lines indicating the respective observation target sites are displayed in gray.

An observation state determination result based on the still image captured at time T1 is reflected in the observation state display map at time T2 later than time T1. The recording control unit 114 acquires an observation state display map M2 displayed on the display device 50 at time T2, and stores it in the auxiliary storage 103 in association with the image (still image) I1 captured at time T1.

For example, if an image of the sixth observation target site (greater curvature in lower body part from above) is captured at time T1, the observation state display map M2 in which the sixth observation target site is displayed as observed is stored in association with the captured image I1.

In this manner, also in this embodiment, a still image and an observation state display map based on the still image can be stored in appropriate association with each other.

### Modification

Information indicating that an observation state display map is updated may be added to the observation state display map stored in association with a captured still image. For example, character information “Updated” is added within an image constituting the observation state display map. Accordingly, it is possible to easily confirm that the stored observation state display map is updated.

### Third Embodiment

In this embodiment, a still image and an observation state display map are stored by screen capturing (screenshot). That is, the still image is stored by screen capturing, and at the same time, the observation state display map is stored.

As described above, an observation state is determined based on a captured still image. Therefore, the result is reflected on the observation state display map after the still image is captured. Thus, if the still image and the observation state display map are stored by screen capturing, an inappropriate observation state display map may be stored.

In this embodiment, in response to an instruction for capturing a still image, display of the screen is temporarily stored, and subsequently, the observation state display map is updated. That is, display of the screen at a time point of the instruction for capturing an image is temporarily stored, and subsequently, at a stage where an observation state display map for the captured image is obtained, the image in a region of the observation state display map is updated and stored. Accordingly, it is possible to store screen information in which the still image and the observation state display map are accurately associated with each other.

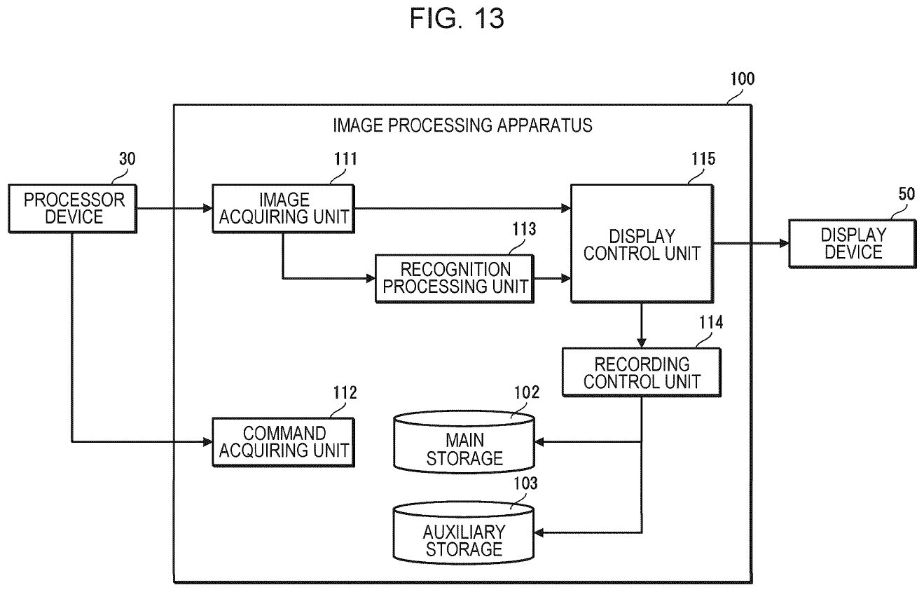

FIG. 13 is a block diagram of main functions of the image processing apparatus.

In response to an instruction for capturing a still image, the recording control unit 114 acquires an image of a screen being displayed on the display device 50 at a time point of the instruction for capturing an image, and stores the image in the main memory 102. In addition, in this embodiment, the image of the screen being displayed on the display device 50 at the time point of the instruction for capturing a still image is an example of a second image. In addition, an image obtained by the endoscope, which is included in the image of the screen, is an example of a first image and an example of second information.

The recognition processing unit 113 includes the observation state determining unit 113C (see FIG. 5). In response to the instruction for capturing a still image, an image of a frame being displayed on the display device 50 at the time point of the instruction for capturing an image is input to the observation state determining unit 113C. Based on an observation state determination result obtained by the observation state determining unit 113C, an observation state display map is generated and displayed on the display device 50.

The recording control unit 114 acquires an image of the generated observation state display map and updates the image of the screen recorded in the main memory 102. That is, an image in part of the observation state display map in the image of the screen is overwritten and rewritten with the image of the newly acquired observation state display map. Then, the image of the updated screen is stored in the auxiliary storage 103. In this embodiment, the observation state display map is an example of first information and third information. In addition, in this embodiment, the main memory 102 and the auxiliary storage 103 are an example of a storage unit.

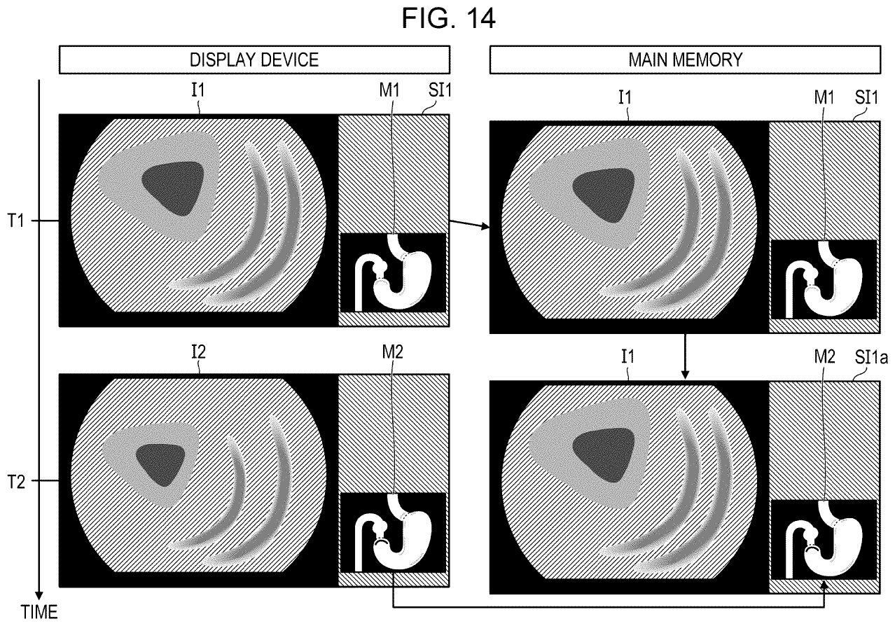

FIG. 14 is a conceptual diagram of storage processing.

It is assumed that an instruction for capturing a still image is issued at time T1. In response to the instruction for capturing an image, an image (screen image) SI1 of a screen being displayed on the display device 50 at that time point (time T1) is stored in the main memory 102. The screen image SI1 includes the image I1 obtained by the endoscope and the observation state display map M1 at time T1. The observation state display map M1 at time T1 does not reflect an observation state determination result for the image I1 at time T1. For example, if still image capturing at time T1 is initial image capturing, the observation state display map M1 displays a state in which all observation target sites are unobserved.

An observation state determination result based on the still image captured at time T1 is reflected in the observation state display map at time T2 later than time T1. The recording control unit 114 acquires an image of the observation state display map M2 displayed on the display device 50 at time T2, and updates the screen image SI1 stored in the main memory 102 with the acquired image. That is, the image of the observation state display map M1 being displayed on the screen image SI1 is overwritten and rewritten with the newly acquired image of the observation state display map M2. Then, an updated screen image (screen image for storage) SI1a is stored in the auxiliary storage 103.

In this manner, also in this embodiment, a still image and an observation state display map based on the still image can be stored in appropriate association with each other.

### Modifications

Display Indicating that Updating is Performed

Information indicating that an observation state display map is updated is preferably added to an image (screen image for storage) in which the observation state display map is updated.

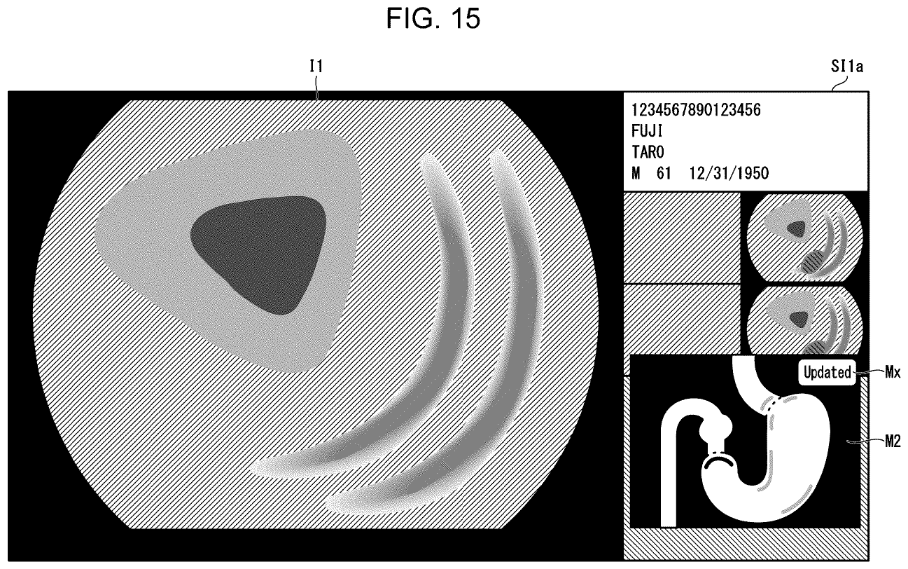

FIG. 15 is a diagram illustrating an example of a screen image for storage in a case where the observation state display map is updated.

As illustrated in FIG. 15, in the screen image SI1a for storage in which the observation state display map M2 is updated, information indicating that observation is completed is added at a predetermined position on the observation state display map M2. In FIG. 15, a mark Mx formed of characters “Updated” is added at an upper right position of the drawing constituting the observation state display map M2 to indicate that the observation state display map M2 is updated. Accordingly, it is possible to easily confirm that the observation state display map M2 is updated, that is, that an observation state determination result is appropriately reflected.

Replacement with Another Image

The above embodiment adopts a configuration in which an image obtained by screen capturing is replaced with an observation state display map that correctly reflects an observation state determination result. However, it is also possible to adopt a configuration in which an image in part of the observation state display map is replaced with another image in the image obtained by screen capturing.

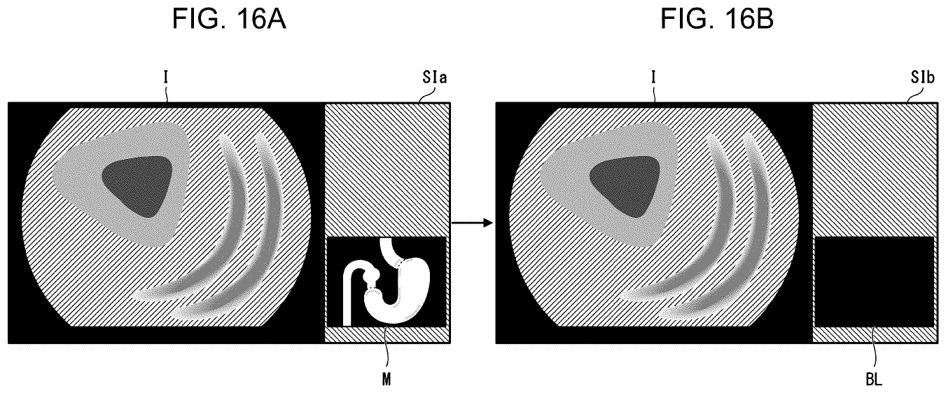

FIGS. 16A and 16B are diagrams illustrating an example of replacement with another image. FIG. 16A is a diagram illustrating an example of an image of a screen being displayed on the display device at a time point of an instruction for capturing a still image. That is, FIG. 16A illustrates a captured screen image. FIG. 16B is a diagram illustrating an example of an image to be stored.

As illustrated in FIGS. 16A and 16B, a display region of an observation state display map is replaced with another image in a screen image obtained by screen capturing. FIG. 16B illustrates an example of a case of replacement with a blank image BL in which no information is displayed. In this case, in response to the instruction for capturing a still image, the recording control unit 114 acquires the image (screen image) SIa of the screen being displayed on the display device 50 at the time point of the instruction for capturing an image, and stores the image SIa in the main memory 102. For this screen image, the recording control unit 114 generates an image (image for storage) SIb in which an image in part of a region of the observation state display map is replaced with the blank image, and stores the image SIb in the auxiliary storage 103. In this example, the image obtained by screen capturing is an example of a second image. In addition, the image obtained by the endoscope, which is included in the image obtained by screen capturing, is an example of a first image. Furthermore, the observation state display map included in the image obtained by screen capturing is an example of first information. In addition, the blank image used for replacement is an example of fourth information. In addition, the screen image in which the image in the region of the observation state display map is replaced with the blank image is an example of a third image.

In this manner, by replacing an image in part of an observation state display map with another image in an image obtained by screen capturing, it is possible to prevent an inappropriate observation state display map from being stored.

Note that although an example of replacement with a blank image has been described in this example, it is also possible to adopt a configuration in which replacement with another image is performed. For example, it is also possible to adopt a configuration in which an image including text or the like is used for replacement.

### Storage of Discrimination Result

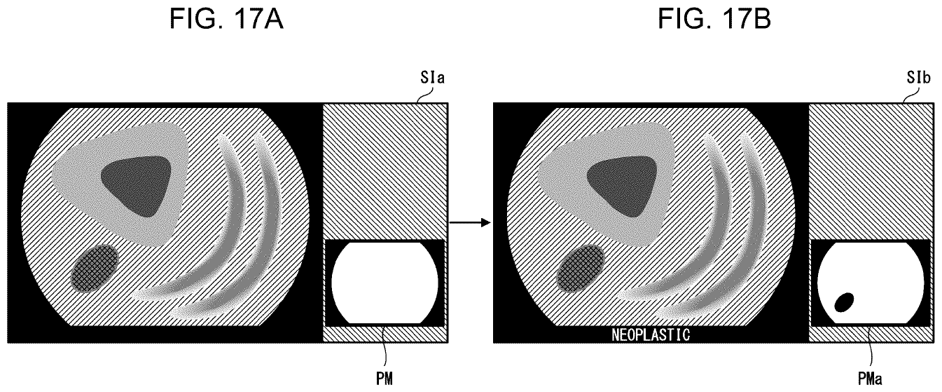

FIGS. 17A and 17B are diagrams illustrating an example of a process in a case where a discrimination result is stored by screen capturing.

FIGS. 17A and 17B illustrate an example of a case where an observation result is displayed on a screen by using a predetermined position map PM. The position map PM is a diagram illustrating the position of a region (lesion part region) that is being discriminated. The region being discriminated is displayed in a color in accordance with a discrimination result. For example, if the discrimination result is neoplastic, the region is displayed in yellow, and if the discrimination result is non-neoplastic, the region is displayed in green.

FIG. 17A is a diagram illustrating an example of the image (screen image) SIa obtained by screen capturing. FIG. 17A illustrates an example of a case where the discrimination result is yet to be output at a time point of screen capturing.

FIG. 17B is a diagram illustrating an example of the screen image (screen image for storage) SIb to be stored. As illustrated in FIG. 17B, the image SIb is generated in which an image in a region of the position map is replaced in the captured screen image SIa. The image used for replacement is an image of a position map PMa based on an image obtained by the endoscope, which is being displayed on the display device at the time point of screen capturing.

Note that also in this example, it is also possible to adopt a configuration in which the position map is replaced with a blank image and stored.

### OTHER EMBODIMENTS

### Information of Processing Result Obtained by Recognizer to be Stored

In the above embodiments, examples of cases in which processing of detecting a lesion part in an image captured by the endoscope, processing of discriminating the detected lesion part, processing of determining an observation state, and the like are performed, and the result is stored. However, the type of recognition processing performed by the image processing apparatus and the type of processing result to be stored are not limited to these. They can be set as appropriate in accordance with an examination purpose or the like.

### Storage Destination

The above embodiments adopt a configuration in which a captured still image, information on a processing result obtained by a recognizer, and the like are stored in an auxiliary storage provided in an image processing apparatus. However, the storage destination of each piece of information is not limited to this. The information can also be stored in an external storage device. For example, it is also possible to adopt a configuration in which the information is stored in a data server or the like connected via a network or the like.

### Hardware Configuration

The functions of the image processing apparatus can be implemented by various processors. Various processors include a central processing unit (CPU) and/or a graphic processing unit (GPU), which are general-purpose processors functioning as various processing units by executing programs, a programmable logic device (PLD), which is a processor in which the circuit configuration is changeable after manufacture, such as field programmable gate array (FPGA), a dedicated electric circuit, which is a processor having a circuit configuration that is specially designed to execute specific processing, such as an application specific integrated circuit (ASIC), and the like. The program is synonymous with software.

One processing unit may be constituted by one of these various processors, or may be constituted by two or more processors of the same type or different types. For example, one processing unit may be constituted by a plurality of FPGAs or a combination of a CPU and an FPGA. In addition, a plurality of processing units may be constituted by one processor. Firstly, as an example of constituting a plurality of processing units using one processor, there is a form in which one processor is constituted by a combination of one or more CPUs and software, and the processor functions as a plurality of processing units, as typified by a computer used as a client, a server, or the like. Secondly, there is a form of using a processor that implements the functions of the entire system including a plurality of processing units with one integrated circuit (IC) chip, as typified by a system on chip (SoC) or the like. In this manner, various processing units are constituted by one or more of the above various processors in terms of hardware configuration.

### REFERENCE SIGNS LIST

1 endoscope system
10 endoscope
20 light source device
30 processor device
31 endoscope control unit
32 light source control unit
33 image processing unit
34 input control unit
35 output control unit
40 input device
50 display device
52 screen
100 image processing apparatus
101 processor
102 main memory
103 auxiliary storage
104 input/output interface
111 image acquiring unit
112 command acquiring unit
113 recognition processing unit
113A lesion detecting unit
113B discriminating unit
113C observation state determining unit
113C1 site recognizing unit
113C2 determining unit
114 recording control unit
115 display control unit
A1 main display region
A2 sub-display region
A3 discrimination result display region
B frame surrounding detected lesion part
BL blank image
I image captured by endoscope
I1 image obtained by endoscope and being displayed on display device at time T1
Ip information on patient
Is captured still image
Ix image (still image) obtained by endoscope and being displayed on display device at time point of instruction for capturing still image
M observation state display map
M1 observation state display map displayed on display device at time T1
M2 observation state display map displayed on display device at time T2
Mx mark indicating that updating is completed
Ot1 first observation target site
Ot2 second observation target site
Ot3 third observation target site
Ot4 fourth observation target site
Ot5 fifth observation target site
Ot6 sixth observation target site
Ox information of observation state determination result
PM position map
PMa replaced position map
SI1 screen image
SI1a screen image for storage
SIa screen image
SIb screen image for storage
Sc schema diagram

What is claimed is:

1. An image processing apparatus that processes images captured in time series by an endoscope, the image processing apparatus comprising:
a recognizer configured to perform recognition processing on the images that are input; and
a processor configured to:
acquire the images in a time-series order;
cause a display unit to display the images in the time-series order;
in response to an instruction for executing the recognition processing, cause the recognizer to perform the recognition processing on, as a first image, an image among the images, which is being displayed on the display unit at a time point of reception of the instruction;
cause the display unit to display first information based on a processing result obtained by the recognizer at a time point displaying an image later than the first image in the time-series order on the display unit; and
store, in a storage unit, third information on the processing result obtained by the recognizer in association with second information for identifying the time point of reception of the instruction for executing the recognition processing.

2. The image processing apparatus according to claim 1, wherein
the instruction for executing the recognition processing also serves as an instruction for capturing a still image, and
the processor is configured to store the first image in the storage unit in response to the instruction for executing the recognition processing.

3. The image processing apparatus according to claim 2, wherein
the second information is constituted by the first image stored in the storage unit in response to the instruction for executing the recognition processing.

4. The image processing apparatus according to claim 1, wherein
the second information is constituted by information of a time or a date and time at the time point of reception of the instruction for executing the recognition processing or information of an elapsed time from start of image capturing.

5. The image processing apparatus according to claim 1, wherein
the processor is configured to:
in response to the instruction for executing the recognition processing, store, in the storage unit in association with the second information, the first information being displayed on the display unit at the time point of reception of the instruction; and
after the processing result of the first image obtained by the recognizer is output, update the first information stored in the storage unit, based on the processing result obtained by the recognizer, and store the updated first information in the storage unit as the third information.

6. The image processing apparatus according to claim 5, wherein
the third information includes information indicating that the first information is updated.

7. The image processing apparatus according to claim 1, wherein
the processor is configured to:
store, in the storage unit, an image that is an image of a screen being displayed on the display unit at the time point of reception of the instruction for executing the recognition processing and that includes the first image and the first information, as a second image; and
after the processing result of the first image obtained by the recognizer is output, by updating part of the first information in the second image stored in the storage unit, based on the processing result obtained by the recognizer, set the updated part of the first information as the third information and store, in the storage unit, the second image including part of the first image as the second information.

8. The image processing apparatus according to claim 7, wherein
the processor is configured to cause the first image to be displayed in a first region set within a screen of the display unit, and the first information to be displayed in a second region set in a region different from the first region.

9. The image processing apparatus according to claim 7, wherein
the third information includes information indicating that the first information is updated.

10. The image processing apparatus according to claim 1, wherein
the first information and the third information are constituted by an image generated based on the processing result obtained by the recognizer.

11. The image processing apparatus according to claim 1, wherein
the first information is constituted by an image generated based on the processing result obtained by the recognizer, and
the third information is constituted by information necessary for generating an image constituting the first information.

12. The image processing apparatus according to claim 1, wherein
the third information is constituted by information necessary for generating the first information.

13. The image processing apparatus according to claim 11, wherein
the third information is information indicating the processing result obtained by the recognizer.

14. The image processing apparatus according to claim 1, wherein
the recognizer is configured to detect a lesion part, and
the first information is constituted by information indicating a position of the lesion part.

15. The image processing apparatus according to claim 1, wherein
the recognizer is configured to discriminate a lesion part, and
the first information is constituted by information indicating a discrimination result of the lesion part.

16. The image processing apparatus according to claim 1, wherein
the recognizer is configured to determine a type of a lesion part, and
the first information is constituted by information indicating the type of the lesion part.

17. The image processing apparatus according to claim 1, wherein
the recognizer is configured to determine a site of a specific organ, and
the first information is constituted by information indicating an observed site in the organ.

18. An image processing apparatus that processes images captured in time series by an endoscope, the image processing apparatus comprising:
a recognizer configured to perform recognition processing on the images that are input; and
a processor configured to:
acquire the images in a time-series order;
cause a display unit to display the images in the time-series order;
in response to an instruction for executing the recognition processing, cause the recognizer to perform the recognition processing on, as a first image, an image among the images, which is being displayed on the display unit at a time point of reception of the instruction;
cause the display unit to display first information based on a processing result obtained by the recognizer at a time point displaying an image later than the first image in the time-series order on the display unit;
acquire, as a second image, an image that is an image of a screen displayed on the display unit at the time point of reception of the instruction for executing the recognition processing and that includes the first image and the first information;
generate, as a third image, an image in which part of the first information in the second image is replaced with fourth information; and
store the third image in a storage unit.

19. An endoscope system comprising:
an endoscope; and
the image processing apparatus according to claim 1.

20. An endoscope system comprising:
an endoscope; and
the image processing apparatus according to claim 18.
